(19) United States
(12) Patent Application Publication
(10) Pub. No.: US 2004/0072728 A1
(43) Pub. Date: Apr. 15, 2004

(54) Use of streptavidin to inhibit transplant rejection

(76) Inventor: Rashid A. Fawwaz, New York, NY (US)

Correspondence Address: Cooper and Dunham LLP, 1185 Avenue of the Americas, New York, NY, 10036, US

(21) Appl. No.: 10/608,841

(22) Filed: Jun. 26, 2003

Related U.S. Application Data
(60) Provisional application No. 60/391,900, filed on Jun. 26, 2002.

(51) Int. Cl. A61K 38/17

(52) U.S. Cl. 514/2

(57) ABSTRACT

This invention provides methods and articles of manufacture for inhibiting the immunological rejection of a transplant in a subject. The instant methods and articles are based on the use of streptavidin for inhibiting such rejection.

This application claims priority of U.S. Ser. No. 60/391,900, filed Jun. 26, 2002, the contents of which are hereby incorporated by reference.

This invention was made with support under United States Government NIH Grant HL 57229. Accordingly, the United States Government has certain rights in this invention.

Throughout this application, various publications are referenced. Full citations for these publications may be found immediately preceding the claims. The disclosures of these publications are hereby incorporated by reference into this application in order to more fully describe the state of the art as of the date of the invention described and claimed herein.

### BACKGROUND OF THE INVENTION

Streptavidin is a 75,000 Dalton protein, similar to egg white avidin in its biotin-binding properties, which forms a very stable noncovalent complex with vitamin D-biotin. Streptavidin differs from avidin in that it lacks carbohydrate and has a lower isoelectric point (1).

Intravenously administered radio-labeled streptavidin and avidin exhibit high localization in the human breast carcinoma MCF-7 in nude mice (2). This finding led to experiments where radiolabeled streptavidin and avidin were tested as tumor imaging agents and their localization was compared to localization in a control inflammatory reaction induced by the subcutaneous inoculation of a sterile foreign body. The localization of radiolabeled streptavidin and avidin was significantly higher in the inflammatory reaction to the foreign body than in the tumor, and was several-fold higher than in all normal tissues.

There is no known nexus between streptavidin and the inhibition of immunological rejection.

### SUMMARY OF THE INVENTION

This invention provides a method for inhibiting the immunological rejection of a transplant in a subject by administering to the subject, at a suitable time, a prophylactically effective amount of stretavidin.

This invention further provides a pharmaceutical composition comprising streptavidin and a pharmaceutically acceptable carrier.

This invention further provides an article of manufacture comprising a packaging material having streptavidin therein, wherein the packaging material comprises a label indicating that the streptavidin is intended for use in inhibiting the immunological rejection of a transplant in a subject.

Finally, this invention provides an article of manufacture comprising a packaging material having therein, either separately or in combination, streptavidin and an anti-lymphocyte antibody, wherein the packaging material comprises a label indicating that the streptavidin and anti-lymphocyte antibody are intended for use in inhibiting the immunological rejection of a transplant in a subject.

### DETAILED DESCRIPTION OF THE INVENTION

Definitions

As used in this application, except as otherwise expressly provided herein, each of the following terms shall have the meaning set forth below.

As used herein, “administering” shall mean delivering in a manner which is effected or performed using any of the various methods and delivery systems known to those skilled in the art. Administering can be performed, for example, topically, intravenously, pericardially, orally, via implant, transmucosally, transdermally, intramuscularly, subcutaneously, intraperitoneally, intrathecally, intralymphatically, intralesionally, or epidurally. “Administering” can also be performed, for example, once, a plurality of times, and/or over one or more extended periods.

As used herein, “antibody” shall include, by way of example, both naturally occurring and non-naturally occurring antibodies. Specifically, ths term includes polyclonal and monoclonal antibodies, and fragments thereof. Furthermore, this term includes chimeric antibodies and wholly synthetic antibodies, and fragments thereof.

As used herein, “inhibiting” the immunological rejection of a transplant shall mean either lessening the likelihood of rejection's onset, or preventing the onset of the rejection entirely. In the preferred embodiment, inhibiting the immunological rejection of a transplant means preventing it entirely.

As used herein, “subject” shall mean any animal, such as a human, non-human primate, mouse, rat, guinea pig or rabbit.

As used herein, “streptavidin” shall include, without limitation, intact streptavidin, biotin-binding fragments thereof, and any artificial or naturally occurring derivatives of intact streptavidin or fragments thereof.

As used herein, “suitable time”, when used in connection with the administration of streptavidin to a transplant recipient, shall mean any time at which streptavidin administered would be expected to inhibit the immunological rejection of the transplant. Suitable times include, without limitation, immediately prior to the transplant procedure (e.g., within one or two days prior), during the transplant procedure, and following the transplant procedure (e.g., at one, two, three, four and/or five days after).

As used herein, “transplant” shall include, without limitation, any organ (e.g., liver, heart, kidney or skin) organ part, tissue, collection of cells (e.g., pancreatic cells or stem cells) or article of manufacture comprising same which is placed into a subject and is thus in contact with the subject's immune system.

Embodiments of the Invention

This invention provides a method for inhibiting the immunological rejection of a transplant in a subject by administering to the subject, at a suitable time, a prophylactically effective amount of streptavidin.

In the preferred embodiment, the subject is a human. The transplant can be, for example, an organ transplant, a tissue transplant, an allogenic transplant, and/or a xenogenic transplant.

In one embodiment of the instant method, the streptavidin is administered intraperitoneally, intravenously or subcutaneously.

The dose for administering to a human subject can be determined according to routine methods based on, for example, animal data, the subject's weight and the route of administration. In one embodiment, the streptavidin is administered in a dose of between about 2 mg/kg to about 200 mg/kg of subject body weight per day. In a further embodiment, the streptavidin is administered in a dose of between about 10 mg/kg to about 40 mg/kg of subject body weight per day. In still another embodiment, the streptavidin is administered in a dose of about 20 mg/kg of subject body weight per day.

The instant method can further comprise the step of administering an antilymphocyte antibody to the subject at a suitable time. In one embodiment, the anti-lymphocyte antibody is administered at a time different from that when streptavidin is administered. In a further embodiment, the anti-lymphocyte antibody is administered in a single dose of about 0.5 ml to about 5.0 ml of solution wherein the antibody is at a concentration similar to that at which it would exist in anti-lymphocyte serum. In still another embodiment, the anti-lymphocyte antibody is administered in a single dose of about 0.2 ml to about 1.0 ml of such solution. In yet another embodiment, the anti-lymphocyte antibody is administered in a single dose of about 0.5 ml of such solution.

This invention further provides for a method of inhibiting the immunological rejection of a transplant which comprises treating the transplant itself with a prophylactically effective amount of streptavidin prior to placing the transplant in a subject.

This invention further provides a pharmaceutical composition comprising streptavidin and a pharmaceutically acceptable carrier.

This invention further provides a first article of manfacture comprising a packaging material having streptavidin therein, wherein the packaging material comprises a label indicating that the streptavidin is intended for use in inhibiting the immunological rejection of a transplant in a subject.

Finally, this invention provides a second article of manufacture comprising a packaging material having therein, either separately or in combination, streptavidin and anti-lymphocyte antibody, wherein the packaging material comprises a label indicating that the streptavidin and anti-lymphocyte antibody are intended for use in inhibiting the immunological rejection of a transplant in a subject. In the preferred embodiment of the first and second articles, the subject is a human.

This invention is illustrated in the Experimental Details section which follows. This section is set forth to aid in an understanding of the invention but is not intended to, and should not be construed to, limit in any way the invention as set forth in the claims which follow thereafter.

Experimental Details

Synopsis

The fact that immune response to acute allograft rejection is mediated by inflammatory cells and that therapeutic doses of non-radioactive streptavidin inhibit the growth of MCF-7 tumor in mice (3) led us to hypothesize that peritransplant administration of streptavidin might prolong graft survival by interfering with immunoreactive cells. Therefore, the effect of a 5-day peritransplant recipient treatment with streptavidin on cardiac allograft survival in the Lewis-to-ACI histoincompatible rat combination was examined.

Treatment of naive ACI recipients with 20 mg/kg i.p. streptavidin alone significantly prolonged Lewis cardiac allografts from a mean survival time (MST±SD) of 9.8±0.7 days in controls to 19.8±6.5 days, with one recipient accepting the graft permanently (>250 days). Peritransplant streptavidin treatment combined with 0.5 ml ALS transient immunosuppression led to permanent graft survival (>250 days) in 6 of 10 recipients. Second-set skin grafts performed 60 days after the primary cardiac allograft were prolonged to 45 days, whereas the third party WF skin grafts were rejected in 15 days without the rejection of the primary Lewis cardiac allografts. Pathology of transplanted cardiac allografts at 100 days showed no mononuclear cell infiltration or chronic allograft vasculopathy. Streptavidin given for 5 days at 20 mg/kg caused a moderate initial weight loss but had no effect on hematologic, biochemical and histologic parameters in the treated recipients.

This study demonstrates that peritransplant recipient treatment with streptavidin combined with peritransplant ALS induces prolonged cardiac and second set skin allograft survival. Therefore, recipient peritransplant streptavidin treatment provides a new strategy for the induction of transplant tolerance.

Materials and Methods

ACI (RT1^{a}) recipients were given intraperitoneal (i.p.) injection of 20 mg/kg Immunopure streptavidin (purchased from Pierce, Rochford, Ill.), dissolved in saline at a concentration of 10 mg/ml, on 5 consecutive days after transplantation, with or without i.p. injection of a single dose of 0.5 ml rabbit anti-rat lymphocyte serum (ALS, Sera Lab, Accurate Chemical, Westbury, N.Y.) on the day of heart transplantation. The ACI control group was pretreated with 0.5 ml i.p. normal saline on the day of cardiac allograft transplantation and continued until post transplant day 5. Other control groups included animals injected with ALS alone or left untreated.

Heterotopic heart transplantation was performed using the modified technique of Ono and Lindsey (4). The cardiac allograft survival was determined by daily palpation and rejection was complete on cessation of a palpable beat. This was further confirmed by histologic examination.

Full thickness (1.5 cm×1.5 cm) skin grafts were obtained from both Lewis and WF donors and transplanted on the ventral aspect of the recipient below the rib cage. Whereas the primary Lewis second set skin graft was placed on the right side of the recipient, the third party WF skin graft was placed on the left. Skin grafts were sutured in place using 4-0 Vicryl suture. A dressing of petroleum gauze, and plaster of Paris for 5 days held grafts in place. Skin grafts were observed daily until there was complete necrosis.

Results

In normal rats, a dose of 20 mg/kg was well tolerated for 5 days causing only a transient loss of body weight (14%) in the first 10 days. The weight loss stabilized by day 14 and had returned to baseline on day 22 of streptavidin administration. Additional doses (>20 mg/kg) resulted in more pronounced weight loss, thus suggesting that weight loss is dose-dependent in rats. Using a dose of 20 mg/kg streptavidin for 5 days in this study there was a posttransplant weight loss of ˜25% in streptavidin-treated recipients from which the animals fully recovered by posttransplant days 25-30. This dose of streptavidin did not result in changes in the hematological and biochemical assays or in tissue histology of the animals.

The survival of Lewis cardiac allografts is summarized in Table I. The difference in allograft survival among the experimental groups was determined using Kaplan-Meyer estimates of the survival curve and the log-rank test. The survival in control (Group I, n=6) untreated ACI recipients was 9.66±0.75 days. Animals receiving transient immunosuppression alone (0.5 ml i.p. ALS) (Group II, n=6) at the time of transplantation showed a prolonged allograft survival time of 16.8±1.3 days. Peritransplant recipient treatment with 20 mg/kg body weight streptavidin for 5 days significantly prolonged allograft survival to MST±SD of 19.8±6.5 days (p<0.001) in 5 of 6 animals while the sixth recipient accepted its cardiac allograft for more than 250 days. The addition of a single peritransplant dose of ALS immunosuppression to peritransplant streptavidin treatment (Group IV) induced permanent cardiac allograft survival (>250 days) in 6/10 animals, which was significantly longer (p<0.006) than in control animals which received ALS alone.

The unresponsive animals (Group IV) were challenged with donor-specific (Lewis) and third-party (WF) skin grafts 60 days after primary cardiac allograft transplantation. Whereas the long-term cardiac allograft recipients rejected the 3^{rd} party skin grafts at 15 days without rejecting the primary Lewis cardiac allografts, they showed significant prolongation of the donor-type skin graft to 45 days (p<0.001). Rejection of the second set donor-type skin graft did not induce rejection of the primary Lewis heart allografts.

A histologic review of transplanted hearts obtained from long-term cardiac allograft recipients at 100 days after transplantation revealed no significant mononuclear cell infiltration in the grafts compared to the native heart. Intracardiac coronary arteries had no perivascular lymphocytic infiltrates and showed no intimal thickening.

Discussion

There are no published studies regarding the toxicity of streptavidin. There is no known explanation for the observed weight loss which is dependent on both dose and duration of streptavidin administration. This may be related to the biotin-binding effect of streptavidin (5) which may affect intracellular metabolism. Parenteral administration of streptavidin has been shown to decrease serum biotin levels which play a critical role in many enzymatic functions (6).

It has now been shown that a short course of streptavidin in the postoperative period leads to significant allograft prolongation. Since it is well known that transient immunosuppression with ALS combined with immunomodulating strategies (7,8) frequently induces immunologic unresponsiveness in rodents, it was examined whether streptavidin treatment combined with a single dose of ALS might prolong graft survival. The results that streptavidin peritransplant treatment combined with ALS induces permanent graft survival in 6 of 10 animals suggest that streptavidin might have immunomodulating effects on the activated T cells that home and accumulate in rejecting cardiac allografts.

The underlying mechanisms of graft prolongation by streptavidin has not yet been determined. These may be dependent on the ability of streptavidin to interfere with cell division as has been demonstrated in bacteria and tumors (9,10). Streptavidin may critically interfere with proliferation of and function of cells essential for immune response. A further explanation put forward by investigators is that streptavidin exhibits structural homology to the Arg-Tyr-Asp-Ser cell adhesion domain of fibronectin and other matrix-associated glycoproteins (11). Thus, streptavidin may bind to the T-cells mediating rejection via this site and abrogate their adhesion-dependent immune function. Further studies have suggested that altered biocompatibility of cells is due to alteration in the surface charge of cells carrying streptavidin (12).

The use of a short course of streptavidin beginning on the day of transplantation and lasting only 5 days is clinically advantageous to control allograft rejection. It has now been shown that animals which show prolonged allograft survival remain immunocompetent as evidenced by their ability to reject third party skin grafts. Histologic studies of cardiac allografts in these animals show no significant accumulation of inflammatory cells within the myocardium. Importantly, the coronary vessels of these grafts at 100 days show no intimal or medial thickening. This finding raises the possibility that streptavidin can prevent the development of chronic allograft vasculopathy which is one of the major problems of clinical heart transplantation.

In conclusion, it is shown for the first time that peritransplant administration of streptavidin prolongs the survival of cardiac allografts.

References

1. Bayer E. A. et al., Isolation and Preparation of Streptavidin, Principles In Enzymology 184, 80-83 (1990).

2. Fawwaz R. A. et al., Radiolabeled Streptavidin: A potential Breast Tumor Imaging Agent, Am Nucl Med Sci 12, 137-141 (1999).

3. Fawwaz R. A. et al., Antiproliferative Effect of Streptavidin in the Treatment of Human Breast Cancer Xenograft (MCF-7) Growing in Nude Mice, Cancer Res 38, 612 (1997).

4. Ono K., Lindsey E. S., Improved Technique of Heart Transplantation in Rats, J Thorac Cardiovas Surg 57, 225-229 (1969).

5. Miller K. and Tausig F., Biotin-binding By Parenterally-Administered Streptavidin Or Avidin, Bioch And Bioph Res Comm 14, 210-214 (1964).

6. Olcese O. P. et al., The Synthesis of Certain B Vitamins by the Rabbit, J Nutr 35, 577-590 (1948).

7. Oluwole S. et al., Selective Lymphoid Ablation II Comparison with Total Body Irradiation on Cardiac Allograft Survival in Rats, Transplantation 28, 492-498 (1979).

8. Oluwole S. et al., Selective Lymphoid Irradiation: Effect of Palladium-109-Labeled Lymphocytes On Rat Cardiac Allograft Survival, Transplantation 40, 146-150 (1986).

9. Zerega B. et al., Avidin Expression During Chick Chondrocyte and Myoblast Development In Vitro and In Vivo: Regulation of Cell Proliferation, J Cell Sci 114 (8), 1473-1482 (2001).

10. Hunt L. and Barker W. C, Avidin-like Domain in an Epidermal Growth Factor Homolog from a Sea Urchin. FASEB 3, 1260-1264 (1989).

11. Alon R. et al., Cell Adhesion to Atreptavidin Via RGD-dependent Integrins, Eur J. Cell Biol 60 (1) , 1-11 (1993).

12. Muzykanatov V. R. et al., Biochem Biophys Acta 1179 (2), 148-156 (1993).

13. Liu Z. et al., Indirect Recognition of Donor HLA-DR Peptides in Organ Allograft Rejection, J Clin Invest 98, 1150-1157 (1996).

What is claimed is:

1. A method for inhibiting the immunological rejection of a transplant in a subject which comprises administering to the subject, at a suitable time, a prophylactically effective amount of streptavidin.

2. The method of claim 1, wherein the subject is a human.

3. The method of claim 1, wherein the transplant is an organ transplant.

4. The method of claim 1, wherein the transplant is a tissue transplant.

5. The method of claim 1, wherein the transplant is an allogenic transplant.

6. The method of claim 1, wherein the transplant is a xenogenic transplant.

7. The method of claim 1, wherein the streptavidin is administered intraperitoneally.

8. The method of claim 7, wherein the streptavidin is administered in a dose of between about 2 mg/kg to about 200 mg/kg of subject body weight per day.

9. The method of claim 8, wherein the streptavidin is administered in a dose of between about 10 mg/kg to about 40 mg/kg of subject body weight per day.

10. The method of claim 9, wherein the streptavidin is administered in a dose of about 20 mg/kg of subject body weight per day.

11. The method of claim 1, wherein the streptavidin is administered intravenously.

12. The method of claim 11, wherein the streptavidin is administered in a dose of between about 2 mg/kg to about 200 mg/kg of subject body weight per day.

13. The method of claim 12, wherein the streptavidin is administered in a dose of between about 10 mg/kg to about 40 mg/kg of subject body weight per day.

14. The method of claim 13, wherein the streptavidin is administered in a dose of about 20 mg/kg of subject body weight per day.

15. The method of claim 1, wherein the streptavidin is administered subcutaneously.

16. The method of claim 15, wherein the streptavidin is administered in a dose of between about 2 mg/kg to about 200 mg/kg of subject body weight per day.

17. The method of claim 16, wherein the streptavidin is administered in a dose of between about 10 mg/kg to about 40 mg/kg of subject body weight per day.

18. The method of claim 17, wherein the streptavidin is administered in a dose of about 20 mg/kg of subject body weight per day.

19. The method of claim 1, further comprising the step of administering an anti-lymphocyte antibody to the subject at a suitable time.

20. The method of claim 19, wherein the anti-lymphocyte antibody is administered to the subject concurrently with streptavidin.

21. The method of claim 19, wherein the anti-lymphocyte antibody is administered to the subject at a time different from that when streptavidin is administered.

22. A pharmaceutical composition comprising streptavidin and a pharmaceutically acceptable carrier.

23. An article of manufacture comprising a packaging material having streptavidin therein, wherein the packaging material comprises a label indicating that the streptavidin is intended for use in inhibiting the immunological rejection of a transplant in a subject.

24. An article of manufacture comprising a packaging material having therein, either separately or in combination, streptavidin and anti-lymphocyte antibody, wherein the packaging material comprises a label indicating that the streptavidin and anti-lymphocyte antibody are intended for use in inhibiting the immunological rejection of a transplant in a subject.

25. The article of claim 23, wherein the subject is a human.

26. The article of claim 24, wherein the subject is a human.
